(19) United States
(12) Patent Application Publication
(10) Pub. No.: US 2011/0172563 A1
(43) Pub. Date: Jul. 14, 2011

(54) Apparatus and Method for Measuring the Dimensions of the Palpable Surface of the Prostate

(75) Inventors: Ian Peter Kellett, Salem, MA (US); John R. Erickson, Middleton, MA (US); Robert Schelsinger, West Sand Lake, NY (US)

(73) Assignee: MEDICAMETRIX, INC., Wayland, MA (US)

(21) Appl. No.: 12/841,485

(22) Filed: Jul. 22, 2010

Related U.S. Application Data
(63) Continuation of application No. 11/943,271, filed on Nov. 20, 2007, now abandoned.
Application No. 11/943,271 is a continuation-in-part of application No. 10/997,779, filed on Nov. 24, 2004, now Pat. No. 7,309,319.
(60) Provisional application No. 60/524,552, filed on Nov. 24, 2003.

(51) Int. Cl. A61B 5/103

(52) U.S. Cl. 600/587

(57) ABSTRACT

A method and system for measuring the size of the palpable surface of the prostate in the physician's office. In one embodiment a membrane with a grid pattern is located proximate the prostate. A physician moves a measurement system, formed at the end of the index finger and located in a glove, between opposite margins of the prostate. The measurement system counts the grid elements between the margins that translate into a quantitative measurement of size of the palpable surface of the prostate. In another embodiment an accelerometer attaches to the end of the index finger to produce signals along multiple axes that a processing system converts into a measurement of the size.

### CROSS REFERENCE TO RELATED APPLICATION

This application is a continuation of co-pending U.S. application Ser. No. 11/943,271 filed Nov. 20, 2007 that is a continuation-in-part of co-pending U.S. application Ser. No. 10/977,779 filed Nov. 24, 2004 and now U.S. Letters Pat. No. 7,309,319 granted Dec. 18, 2007 that claims priority from U.S. Provisional Application No. 60/524,552 filed Nov. 24, 2003 for Apparatus and Method for Measuring the Dimensions of the Palpable Surface of the Prostate.

### BACKGROUND OF THE INVENTION

1. Field of the Invention

This invention relates generally to medical devices. It relates particularly to a method and system for use by a physician for measuring the palpable surface of the prostate thereto to determine the size and growth of the prostate and more specifically to a method and apparatus for the enabling trans-rectal measurement of the size, growth rate and changes in growth rate of the prostate during a standard digital rectal exam.

2. Description of Related Art

Prostate gland, or prostate, problems are widespread in the male population, especially the older male population. In particular, benign prostatic hyperplasia (BPH) and prostate cancer are common in men over 50 years of age. Indeed, prostate cancer is the second most common cancer in men in this country. Each year there are over 200,000 new cases and over 30,000 deaths. However, if prostate cancer is detected early and treated effectively, the chance of survival improves significantly. Unfortunately, conventional methods for detecting prostate problems are wanting as many early stage cancers go undetected.

In an attempt to enhance the efficiency and efficacy of methods and systems of detection of prostate cancer, medical science has used ultrasonics to diagnose prostate problems. Such systems are very expensive, and are not yet widely available in the urologist's or primary care physician's examining room. Most ultrasound imaging is performed by radiologists at an outside facility, or at the practitioner's office on a contract basis with a portable ultrasound unit. The technology and interpretation is difficult to master, requiring a time-consuming learning curve. Consequently, no routine examining system or technique exists which provides the high degree of accuracy in monitoring prostatic growth, nor is the required repeatability of results achieved.

Thus, the digital rectal examination continues to be the modality of choice for monitoring the prostate even though the process is very subjective. The standard exam is done by inserting a finger into the rectum and palpating or feeling the palpable surface of the prostate. The physical characteristics of the prostate size, contour, consistency, symmetry, presence or absence of the lateral margins, and the presence or absence of nodularity, are assessed and recorded by attempting to translate the physician's subjective impressions into a written record. This method of data collection is inexact and makes comparisons from exam to exam very difficult.

What is needed is a method and apparatus that overcomes these deficiencies by enabling the physician to monitor changes in the size of the palpable surface of the prostate for a given patient over time thereby to gauge more accurately changes in the size of the prostate.

### SUMMARY

Therefore it is an object of this invention to provide a method and apparatus for facilitating examinations of the prostate.

Another object of this invention is to provide a method and apparatus for providing quantitative information concerning the size of the prostate.

Still another object of this invention is to provide a method and apparatus for providing repeatable quantitative information about the size of the prostate.

Yet another object of this invention is to provide a method and apparatus for examining the prostate that is simple to use.

Still yet another object of this invention is to provide a medical device for facilitating digital examination of a patient's prostate that provides repeatable quantitative information that is easy to use and that is readily manufactured.

Still yet another object of this invention is to provide a method of making accurate and repeatable quantitative measurements of the size of the palpable surface of the prostate thereby to establish a basis for calculating prostatic volume, rate of growth and changes in rate of growth, and a method for storing the resultant data in a database for subsequent recovery and comparison. It is used at the same time, and in conjunction with, a digital examination, allowing the physician to gather the traditional subjective data but with the added capability of accurate measurements of the prostate being palpated.

Still yet another object of this invention is provide a method of measuring the size of the palpable surface of the prostate using a motion transducer during a conventional digital rectal examination. In accordance with one embodiment of this invention, a medical device provides an input to a signal processor that generates data representing the size of the palpable surface of a patient's prostate during a digital rectal examination by a physician. The medical device comprises a motion transducer for generating displacement dependent signals during palpitation of the prostate. A transducer positioner attaches the motion transducer to the end of one of the physician's fingers. Signals from the motion transducer means are coupled to the processor to provide data representative of the size of the palpable surface of the prostate.

In accordance with another aspect of this invention a physician can measure changes in the size of a prostate by measuring the size of the palpable surface of the prostate during digital examinations. A closed volume is formed within the rectum proximate the prostate and about the physician's index finger. Motion of the physician's index finger is converted into displacement signals. Processing the displacement signals yields a measurement of the size of the palpable surface of the prostate that is a function of the size of the prostate.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 is a schematic view of a prostate measurement system embodying this invention;

FIG. 2 shows a cross-section through a sensor and membrane shown in FIG. 1 useful in understanding this invention;

FIG. 3 is a plan view of a portion of the prostate measurement system shown in FIG. 2;

FIG. 4 is an enlarged cross-section of an index finger structure of the prostate measurement system;

FIG. 5 is a perspective view, partially cut away, of a membrane with a grid that is useful in accordance with this invention;

FIG. 6 is a plan view, partially broken away, of a glove useful in accordance with this invention;

FIG. 7 is a view of another embodiment of this invention;

FIG. 8 is a view an enlarged cross-section the apparatus in FIG. 7;

FIG. 9 is a plan view of a portion of the apparatus shown in FIGS. 7 and 8; and

FIG. 10 is of another embodiment of the apparatus shown in FIGS. 7 through 9.

### DESCRIPTION OF ILLUSTRATIVE EMBODIMENTS

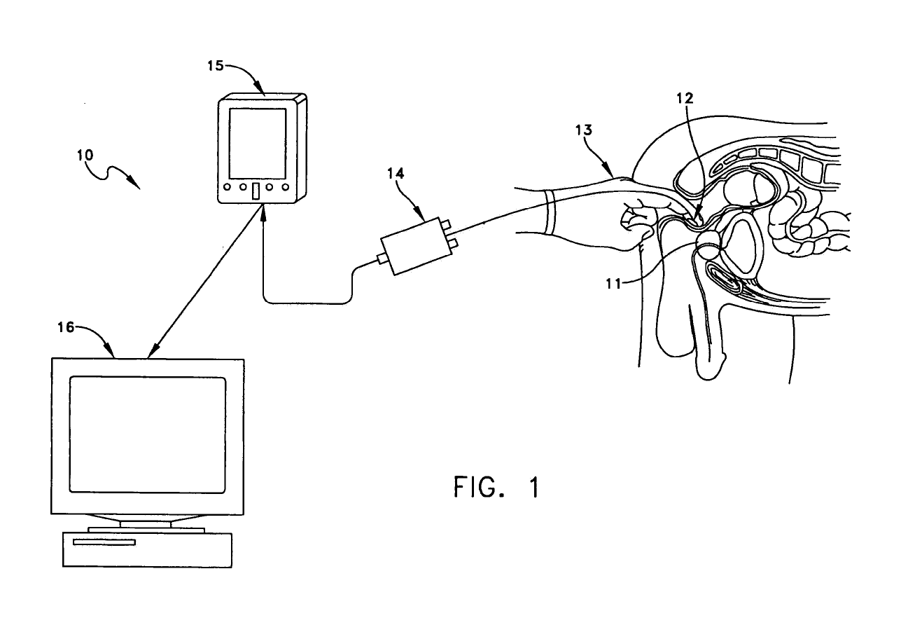

In FIG. 1 a system 10 in accordance with this invention allows a physician to measure the size of a patient's prostate during a digital rectal examination. More specifically, a physician positions a sensor 12 in the rectum proximate to the prostate 11 during a standard digital rectal exam. The sensor 12 includes a motion transducer for generating displacement dependent signals during palpitation of the prostate. The sensor 12 attaches to the physician's index finger and connects through an interface 14 to a computing device such as a personal digital assistant (PDA) 15 which in turn can communicate with a workstation and/or server 16 for storing the data for future reference. The computing device, or processor, converts the displacement dependent signals to data representing the size of the palpable surface of the patient's prostate. A disposable glove 13 overlies the physician's index finger and the sensor 12 thereby to attach the sensor 12 to the end of one of the physician's fingers, typically the index finger.

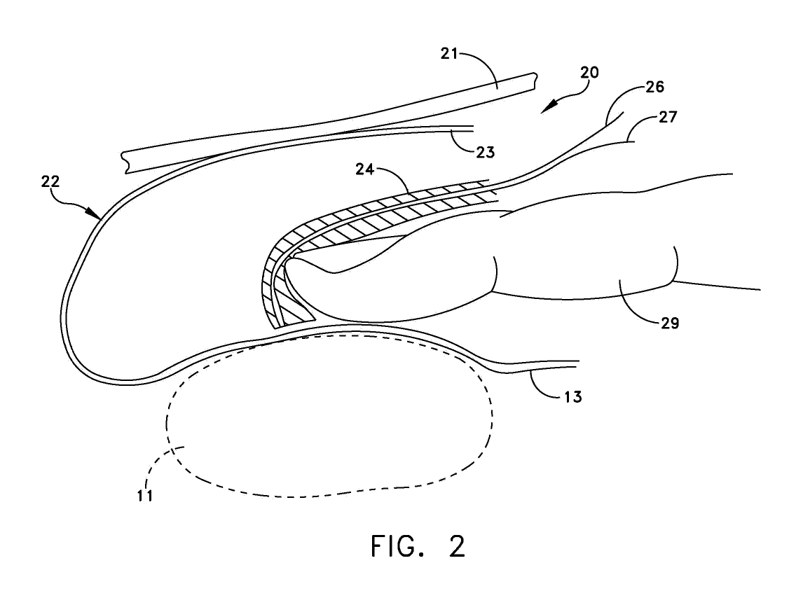

Referring to FIG. 2, a patient's rectum 20 is bound by a rectum wall 21 which acts as a barrier between the rectum 20 and the prostate 11. The apparatus and method for performing an examination in accordance with this invention includes a closed membrane or sac 22 formed by a loose fitting forefinger 23 of the disposable glove 13 and a reusable index finger structure 24. As shown in FIGS. 2, 3 and 4, the index finger structure 24 is formed of a flexible material that overlies the end portion and the top of the physician's index finger. In this particular embodiment two optical fibers 26 and 27 and a passage or duct 30 are embedded in the index finger structure 24. A saddle 28 or similar clamping or attaching structure affixes the index finger structure 24 on the physician's forefinger 29.

The optical fiber 26 connects to a light source 31, generally located in the interface 14. The optical fiber 27 connects to a photodetector 32 in the interface 14. The fibers 26 and 27 thereby couple the motion transducer, specifically the sensor 12, to the processing means, specifically the PDA 15. An air hose 33 connects the duct 30 to a source of low-pressure air 34.

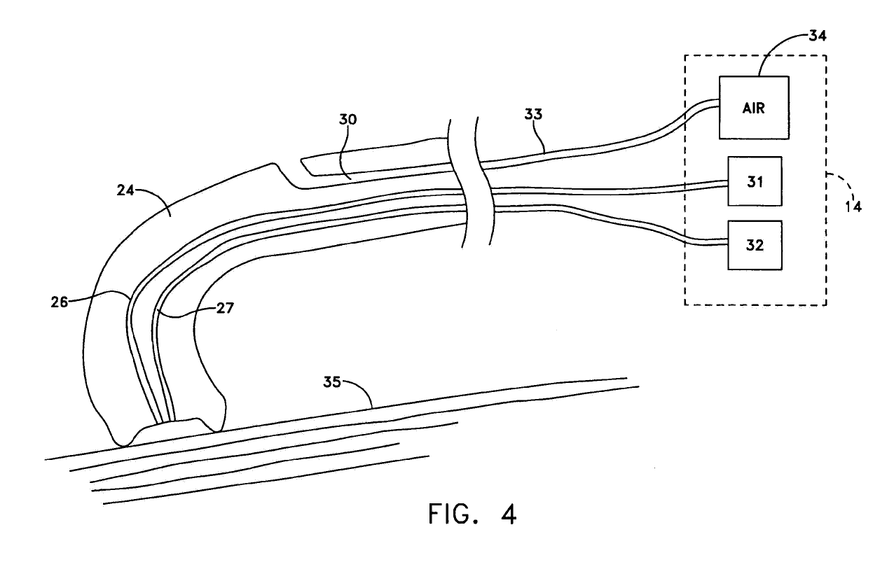

Referring again to FIGS. 2 and 4, for an examination a physician places the index finger structure 24 on the index finger 29 and then dons the glove 13 incorporating the membrane 22 of the forefinger 23 over the index finger structure 24. The physician inserts the index finger into the rectum and positions the membrane 22 proximate the prostate 11.

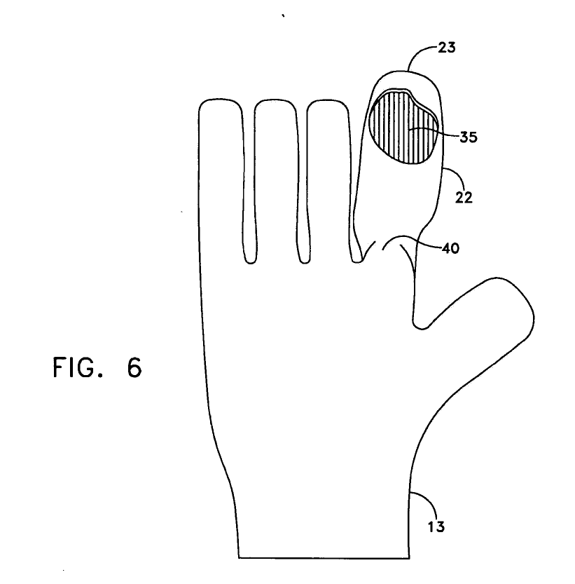

Low-pressure air is then used to inflate and press the membrane 22 against the surrounding portions of the rectum wall 21 particularly in close contact with portions of the rectum wall 21 that overlie the palpable area of the prostate 11. A sealing structure in the forefinger 23, as shown in FIG. 6, confines the increased air pressure to the volume of the membrane 22. As will be apparent, when inflated, the membrane 22 defines a closed, clean volume within the rectum 20 proximate the prostate 11. Also the inflation of the membrane 22 by the air source 34 in FIG. 1 stabilizes position of the membrane 22 proximate the prostate 11.

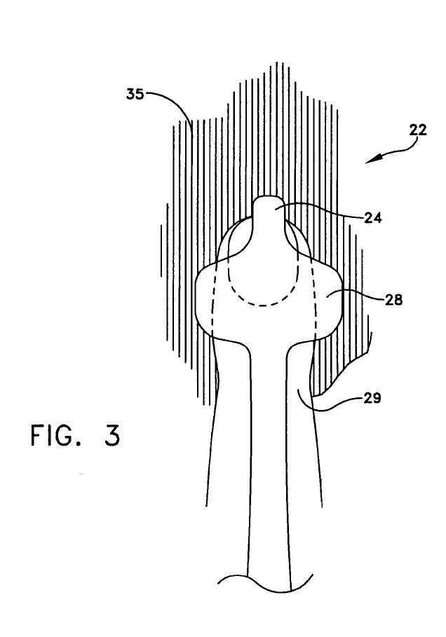

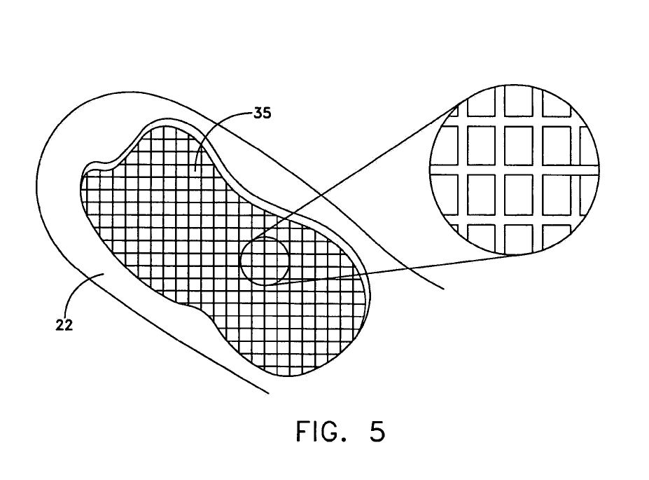

Referring now to FIGS. 3 and 5, motion transducer includes a reference pattern on the membrane 22 in the form of a grid 35 which is visible from the interior of the membrane 22. The specific grid 35 in FIG. 3 has parallel lines printed on the interior of the membrane 22 in a contrasting color with respect to the membrane material. Alternatively the printing could include contrasting colors. In this embodiment the grid defines closely spaced, alternating light and dark lines parallel lines approximately parallel the physician's finger to enable lateral measurement, i.e., from left to right or vice versa in FIG. 3.

As an alternate, the grid pattern could include a set of alternating dark and light lines at right angles to the set shown in FIG. 3. Still another pattern could include a combination of both sets to provide intersecting lines. Other alternative grid arrangements are also possible as, for example, a pseudo-random pattern of small dots or a pattern of connected hexagons. In any of these configurations the grid 35 provides a reference pattern over the palpable surface of the prostate.

In use, a physician moves the finger structure 24 over the grid 35 to transducer that motion into displacement dependent signals during the palpitation thereby to determine the area of contact between the rectum wall 21 and the prostate 11. The areas where the prostate 11 separates from the rectum wall 21 are called “margins”. Physicians can locate margins accurately and with good repeatability. After a physician locates one margin, the physician enables, by a switch or other means, the measurement process and moves the index finger to the opposite margin.

As the interior of the membrane 22 is clean, light reflects back from the grid to the photodetector 32 in FIG. 4 to produce displacement dependent signals as the index finger structure 24 moves past the grid 35 during palpitation thereby changing the light reflected to the photodetector 32. The PDA 15 in FIG. 1 or another equivalent device counts the number of lines in the grid that have been traversed using these displacement signals. When the physician reaches the other margin, the physician disables the system to complete the measurement. At this point the PDA 15 or other device has recorded the number of counts and may include the necessary software for providing a desired measurement of the distance traveled by the index finger structure 24 and sensor 12. This can be converted into a measurement of the size of the palpable surface of the prostate along the direction of sensor motion.

As an alternative to manual switching, the information in the signal may provide automatic delimiters on the measurement. Moreover, the PDA or related device 15 may perform all the processing that will use this information to determine the volumetric size of the prostate. Alternatively, measurements can be downloaded to a data processing system 16 for further processing and/or permanent storage.

Over time a given patient will have a series of quantitative measurements which can be reviewed by the physician. Changes in these measurement can then provide an indication concerning whether growth of the prostate is normal or abnormal.

The grid pattern on the membrane 22, such as the grid pattern 35, the light source 31 and photodetector 32 constitute on embodiment of a motion transducer that generates the displacement signals in response to the motion of the physician's index finger across the grid pattern.

An alternate index finger structure could include the structures described proximate the index finger in a finger cot as an alternative embodiment of a glove means. The optical fibers could be eliminated by attaching the light source 31 and the light sensor 32 directly to the finger of the physician. In this embodiment the electrical energy for the light source 31 and the electrical signals from the photodetector 32 are connected by insulated wires to the interface circuit.

In another embodiment of the invention, the distance across the surface of the prostate is measured using a roller, ball or wheel which rolls across the surface to be measured and the distance traveled is expressed in terms of the number of degrees of rotation of the wheel or ball. The roller, ball or wheel is affixed to the end of the physician's finger and the rotation monitored by photo-electric or electro-magnetic sensors.

In still another embodiment, a pattern of energizable wires or conducting filaments embedded in the thickness of the disposable glove could be substituted for the grid pattern. In this embodiment, the index finger structure would include an electrostatic or electromagnetic sensor. Alternatively, the function of the filaments and the sensor might be reversed.

In still another embodiment, the printed grid pattern could be replaced with a magnetic coating encoded by a magnetic pattern. A playback head could then be attached to the index finger structure.

Each of these embodiments provides a medical device that facilitates examinations of the prostate and the correlation of quantitative data about the size of the palpable surface of the prostate related to successive examinations of a single patient.

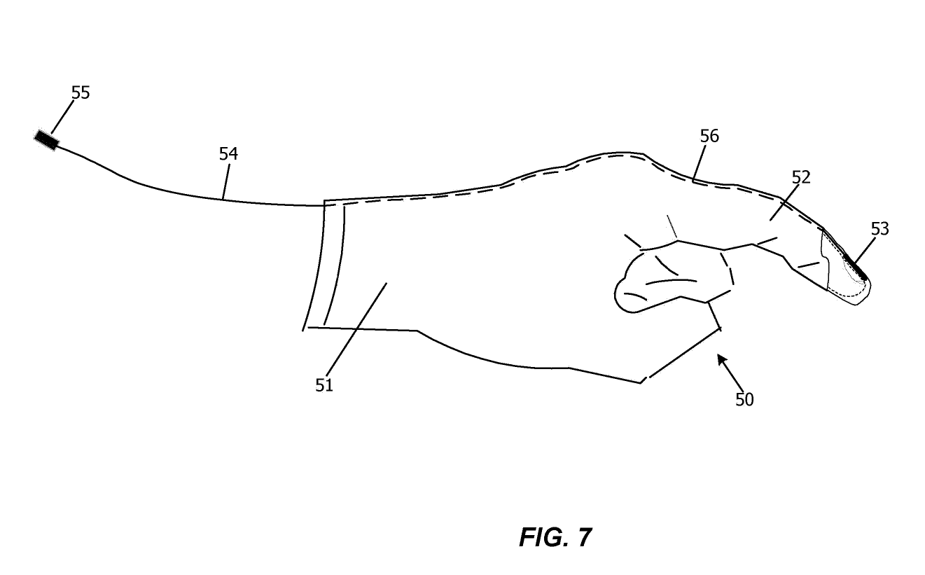

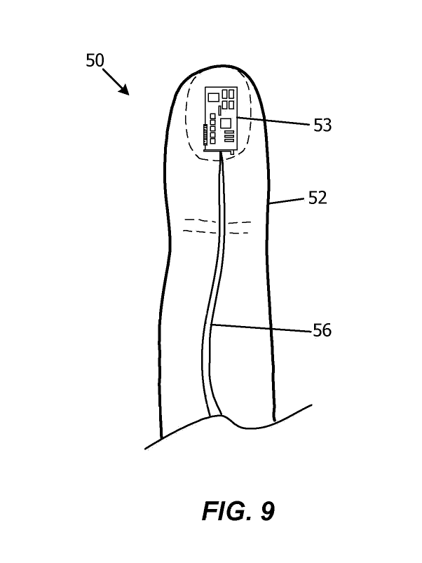

FIGS. 7 through 10 depict other embodiments of this invention in which a system 50 enables a physician to make a measurement of the size of the palpable surface of a patient's prostate 11 during a digital rectal examination. The embodiment of FIGS. 7 through 9 includes a glove 51 including an index finger covering portion 52 with an integral motion transducer in the form of an accelerometer 53 embedded in the glove portion 52 proximate the end of the physician's index finger. In some cases and as previously described, a physician may use a glove means constituted by a finger cot. The structure would be the same as described with respect to FIGS. 7 through 9.

Conductors 54 couple the accelerometer 53 to a connector 55 that attaches to a processing system that may take the form of the processing system shown in FIG. 1. Portions 56 of the conductors 55 are also embedded in the glove. In this embodiment the glove positions the accelerometer with respect to the physician's index finger. Consequently, as the physician moves an index finger during palpitation, the accelerometer 53 tracks the motion accurately.

Several versions of accelerometers are available commercially that include an integral sensor and preprocessor for converting the signals from the sensor into signals that are compatible with the input to an external processor to maximize data sampling quality. Preferably any such accelerometer 53 will be small, require low voltage and power, be stable electrically and thermally, and operate with multiple sensing axes. For example, measurements could be obtained using an accelerometer with at least three sensing axes. A preferred embodiment includes an accelerometer for generating position dependent signals along four sensing axes; i.e., signals that indicate acceleration in orthogonal X, Y, Z axes and the roll axis. In whatever form, the conductors 54 couple the signals from the preprocessor in the accelerometer to external processing system represented by the connector 55 and analogous to the PDA 15 and/or server 16 in FIG. 1.

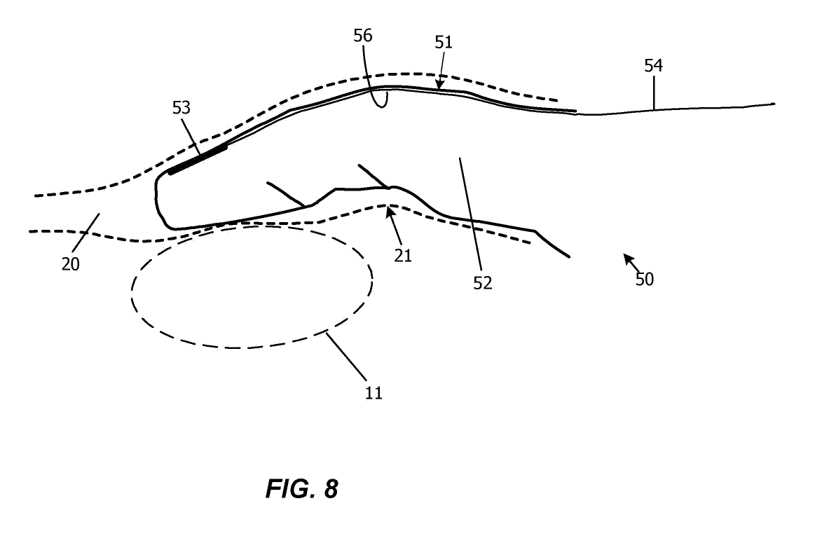

The method for use of this embodiment of the system 50 is best explained by reference to FIG. 8 that depicts a patient's prostate 11 and rectum 20 with the rectum wall 21 intermediate the physician's finger and the prostate 11. Like the procedure described with respect to the medical device shown in FIGS. 1 through 7, the physician dons the glove 51 thereby to position the accelerometer 53 over the end of the index finger and begins the examination by determining the area of contact between the rectum wall 21 and the prostate 11 thereby to determine the “margins.” In one embodiment, the signals at the connector 44 in FIG. 7 may have no relevance as the physician starts the procedure. However, the physician can enable the system to begin recording the accelerometer output once the margins have been located.

Then the physician moves his or her index finger between the margins. The accelerometer produces the output signals. In a preferred embodiment these signals track the motion of the accelerometer. The X-Y signals define the motion components in the X-Y plane components while the Z signals define the motion in the Z direction which is orthogonal to the X-Y plane. The roll signals provide a basis for compensating variations in the other signals based upon the rotation of the physician's finger.

The processor, such as the PDA 15 or server 16, processes the signals from the accelerometer 53 by a double integration technique, as known, to produce a three-dimensional map of the prostate between the margins. This map identifies lateral asymmetry as well as measures the distance across the palpable prostate surface. If the accelerometer generates only X-Y and roll signals, then only the measurement across the palpable surface will be obtained. With either version, the system 50 provides repeatable quantitative measurements that were not otherwise available to the physician during a conventional digital rectal examination. It is also possible to eliminate the need for the physician to initiate processing. Additional signal processing of all the signals from the accelerometer can be processed further to isolate those signals of interest.

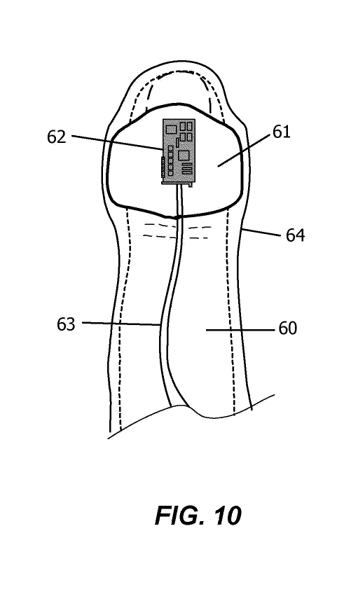

FIG. 10 depicts an alternative embodiment to that shown in FIGS. 7 through 9 that allows a physician to use a system of this invention in combination with a conventional surgical glove. In this embodiment, the medical device attaches to a physician's index finger 60 by means of a finger structure 61 that clamps to the finger and that is formed with an integral accelerometer 62. After coupling wires 63 are routed back along the finger and hand, the physician dons a conventional glove 64 covering the index finger, the accelerometer 53 and the coupling wires 63. The diagnostic procedure remains the same.

Each embodiment disclosed in FIGS. 1 through 10 provide repeatable quantitative measurements so a physician can monitor prostate growth more accurately, including such parameters as rate of growth and changes in the rate of growth. Further, these measurements can be made during the course of a standard digital examination without hindering physician's ability to obtain the subjective information about the prostate that the physician normally obtains. Each embodiment enables the measurements to be made efficiently. Moreover, it will be apparent that the specifically disclosed embodiments can be readily manufactured using standard techniques.

As will now also be apparent, this invention can be implemented in a variety of configurations. Each of the foregoing or other modifications could be made while attaining some or all of the advantages of this invention. Thus, it is the intent of the appended claims to cover all such variations and modifications as come within the true spirit and scope of this invention.

What is claimed as new and desired to be secured by Letters Patent of the United States is:

1. A medical device for attachment to a signal processor that generates, in response to an input, data representing the size of the palpable surface of a patient's prostate during a digital rectal examination by a physician, said medical device comprising:
A) motion transducer means for generating displacement dependent signals during palpitation of the prostate,
B) positioning means for attaching said motion transducer to the end of one of the physician's fingers, and
C) coupling means for connecting said motion transducer means to the processor whereby signals from said motion transducer means are coupled to the processor to provide data representative of the size of the palpable surface of the prostate.

2. A medical device as recited in claim 1 wherein said motion transducer means comprises accelerometer means for generating signals dependent upon the motion thereof.

3. A medical device as recited in claim 2 wherein said accelerometer means generates signals for multiple axes of sensing.

4. A medical device as recited in claim 2 wherein said accelerometer means generates signals for at least three axes of sensing.

5. A medical device as recited in claim 2 wherein said accelerometer means includes an accelerometer X, Y, Z and roll axes of sensing.

6. A system for use by a physician for making a measurement of the size of the palpable surface of a patient's prostate during a digital rectal examination, said medical device comprising:
A) motion transducer means for generating displacement dependent signals during palpitation of the prostate,
B) positioning means for attaching said motion transducer to the end of one of the physician's fingers,
C) processing means for converting the displacement signals from said motion transducer means to data representative of the size of the palpable surface of the prostate, and
D) coupling means for connecting said motion transducer means to said processing means.

7. A system as recited in claim 6 wherein said positioning means comprises glove means for covering at least the one physician's finger, said motion transducer means being affixed to said glove means proximate the end of the one finger.

8. A system as recited in claim 7 wherein said motion transducer means is embedded in said glove proximate the end of the physician's index finger.

9. A system as recited in claim 6 wherein said positioning means comprises attachment means affixed to said motion transducer means for being attached to the end of the one finger.

10. A system as recited in claim 9 wherein said motion transducer means is attaches to the physician's index finger proximate the end thereof.

11. A system as recited in claim 6 wherein said motion transducer means comprises accelerometer means for generating signals dependent upon the motion thereof.

12. A system as recited in claim 11 wherein said accelerometer means generates signals for multiple axes of sensing.

13. A system as recited in claim 11 wherein said accelerometer means generates signals for at least three axes of sensing.

14. A system as recited in claim 11 wherein said accelerometer means includes an accelerometer X, Y, Z and roll axes of sensing.

15. A method by which a physician can measure the size of the palpable surface of a prostate during a digital rectal examination comprising:
A) positioning the physician's index finger within the rectum proximate the prostate,
B) converting motion of the physician's index finger into displacement signals, and
C) processing the displacement signals from said motion transducer means into a measurement that of the size of the palpable surface of the prostate.

16. A method as recited in claims 15 wherein said conversion includes locating an accelerometer for motion with the physician's index finger while the accelerometer is overlaid with a portion of a glove.
